(19) United States
(12) Patent Application Publication
(10) Pub. No.: US 2003/0049866 A1
(43) Pub. Date: Mar. 13, 2003

(54) Sample device preservation

(75) Inventors: Paul Bushway, San Diego, CA (US); Laurence Warden, Poway, CA (US); Todd Peterson, Coronado, CA (US); David E. Kohne, La Jolla, CA (US); Juan Yguerabide, La Jolla, CA (US)

Correspondence Address: Wesley B. Ames, FOLEY & LARDNER, P.O. Box 80278, San Diego, CA, 92138-0278, US

(73) Assignee: Genicon Sciences Corporation

(21) Appl. No.: 09/948,058

(22) Filed: Sep. 5, 2001

(51) Int. Cl. G01N 33/543; B05D 3/00

(52) U.S. Cl. 436/518; 427/2.11

(57) ABSTRACT

A method for archiving sample devices such as microarray slides and membranes is described using an optically clear, solidifying solution. Also described are related methods and kits.

### BACKGROUND OF THE INVENTION

The present invention relates to the field of analyte assays using detectable labels, with particular application to assays using light scattering particle labels and to preservation of labeled samples.

The following background description is provided solely to assist the understanding of the reader. None of the information provided herein is admitted to be prior art to the present invention.

The use of detectable labels in a large variety of analyte assays is well known. Such labels include, for example, chromogenic labels, radioactive labels, chemiluminescent labels, fluorescent labels, light absorbing labels, and light scattering labels. Labels may also include enzymatic or non-enzymatic labels for direct or indirect detection of analytes. In many applications, photodetectable labels are preferred.

Commonly, for labeled samples on a solid phase or membrane sample device, the sample must be handled with care to avoid surface damage or other degradation. This is particularly the case where it is desired to delay reading of signal from the device until some later time or to obtain repeat readings at a later time(s). However, many types of labels are not amenable to repeated readings and/or delayed readings due to changes in the label itself. For example, fluorescent labels are subject to bleaching and fading, limiting or eliminating the ability to obtain reproducible repeat readings or reliable delayed readings. Likewise, commonly used radiolabels have relatively short half-lives, limiting the ability to delay reading of labeled samples.

In contrast, resonance light scattering (RLS) particle labels, particularly metal particle light scattering labels, are not subject to such degradation, and can be reproducibly subjected to repeated readings and can provide reliable and accurate delayed readings. Such RLS particle labels and their use, especially in analyte assays, are described in Yguerabide et al. U.S. Pat. U.S. Pat. 6,214,560, PCT/US/97/06584 (WO 97/40181 and Yguerabide et al., PCT/US98/23160 (WO 99/20789), all of which are incorporated herein by refererence in their entireties, including drawings. Elements of the technology are also described in two related articles by Yguerabide & Yguerabide, (1998) Anal. Biochem. 261:157-176; and (1998) Anal. Biochem. 262:137-156, which are likewise incorporated herein by reference in their entireties. In the Yguerabide methods using RLS particle labels, the detection and/or measurement of the light-scattering properties of the particle is correlated to the presence, and/or amount, or absence of one or more analytes in a sample. Such methods include detection of one or more analytes in a sample by binding those analytes to at least one detectable light scattering particle, with a size preferably smaller than the wavelength of the illumination light. This particle is illuminated with a light beam under conditions where the light scattered from the beam by the particle can be detected by the human eye with less than 500 times magnification. The light that is scattered from the particle is then detected under those conditions as a measure of the presence of those one or more analytes. By simply ensuring appropriate illumination and ensuring maximal detection of specific scattered light, an extremely sensitive method of detection can result.

Methods utilizing light scattering (referred to as “plasmon resonance”) labels for assays are also described in Schultz, et al, PCT/US98/02995 (WO 98/37417) and U.S. Pat. No. 6,180,415/Method and apparatus described in the Schultz et al. references can also be used in the present invention.

Samples of other types have been preserved in a variety of ways. For example, stained tissue samples on microscope slides have been coated or embedded in a clear material. Such preserved samples have commonly been used for classroom use to allow a number of different individuals to utilize the sample over a period of time. However, such samples are not generally used to provide quantitative results, but rather are used for qualitative microscopic inspection and teaching.

Likewise, in electron microscopy, it is common to embed a sample in a solid matrix prior to sectioning and inspection.

In yet another example, agarose or polyacrylamide gels containing stained sample are often dried to provide a semi-permanent record of electrophoresis results. However, such drying typically introduces significant distortions as the gel dimensions change during the drying process.

In many circumstances involving detectable labels that specifically associate with a particular material, it is useful to be able to preserve the labeled sample. For example, in many situations, it is beneficial to be able to compare results for samples assayed at different times.

However, the ability to carry out such comparisons have been limited because of instabilities of the sample, instabilities of the assay apparatus, and/or instabilities of the sample device (sample carrier). Likewise, it is often beneficial to be able to carry out repeated detection of signal from a sample device for a variety of other reasons, or to carry out detection of signal after some extended period of time instead of essentially immediately. For these applications also, the ability to perform repeat or delayed detection has been limited by the various instablilities.

Thus, it would be highly advantageous to have a methods and materials that would assist in preserving, protecting, and/or enhancing detection for labeled samples.

### SUMMARY OF THE INVENTION

The present invention addresses the needs for labeled sample protection, preservation, and repeat or delayed detection as well as other advantages and applications by providing methods and materials for preserving samples on sample devices in a manner that provides for such repeated or delayed detection, even after storage for extended periods of time. In addition, when used in conjunction with resonance light scattering particles (RLS particles), the method can also enhance the sensitivity of analyte assays by reducing background scattered light and/or by refractive index enhancement of the scattered light signal. The protection and/or preservation can also be referred to as “archiving”.

Thus, in a first aspect, the invention provides a method for preserving a sample device that has light scattering particle labels attached to it, preferably by coating at least a portion of the sample device with an optically clear, solidifying solution.

However, the sample device can also be preserved using other techniques, for example, by covering at least a portion of the device with a solution that is itself covered with a small optically clear plate, e.g., a plastic, glass, or quartz crystal coverslip or the like. The small plate can be held in place with by surface tension of the solution and/or viscosity of the solution (the solution can act effectively as a glue). The solution may have high viscosity both before and after application (though still sufficiently fluid to cover the sample device without voids, or may become more viscous following application on the sample device. Likewise, at least a portion of the sample device can be covered with a solution that sets up to form a network or gel, for example, polyacrylamide and agarose gels. The network or gel can be covered by a small plate as described above. The plate can be held in place via surface tension and/or by some degree of bonding between the plate and the network or gel.

For embodiments in which a non-solidifying solution is used, the preservation may be shorter term than for embodiments in which a solidifying solution is used, due to drying (especially around the edges of a covering plate). However, in such cases, the preservation can be extended by sealing the non-solidifying solution, thereby significantly slowing the evaporation rate (i.e., reducing the evaporation rate by at least 50%, 70%, 80%, 90%, 95%, or more as compared to the non-sealed case) or effectively stopping evaporation (e.g., slowing the evaporation rate to less than 5%, 3%, 2%, 1%, 0.5% or even less as compared to the non-sealed case). Such sealing can involve covering the non-solidifying solution (and the covering plate if present) with a layer of an optically clear material with low permeability to the solvent or solvents that would otherwise evaporate from the solution to produce the reduced evaporation rate. Alternatively, in cases where the non-solidifying solution is covered with a small plate, the seal maybe only around the edges of the plate. In this case, the sealing material may be, but need not be, optically clear.

A variety of different sample devices can be utilized. Generally, such sample devices provide a sample surface or volume where the labeled material can be surrounded with a preserving solution. Thus, in preferred embodiments, the sample device includes a solid phase array. In preferred embodiments, the sample device includes a slide, an array chip, a microtiter plate, a membrane, or the like.

In preferred embodiments, the method also involves storing the sample device, preferably under dark conditions. Such dark conditions, for example, storage of a sample in a slide box, are commonly recognized to reduce or eliminate light-induced degradation of materials, especially UV light induced degradation.

The sample devices, e.g., as mentioned above, can be configured and/or have samples selected for specific types of applications. In certain applications it is particularly advantageous to be able to preserve sample devices. Thus, in preferred embodiments, the sample device is a forensic sample device or an identification sample device or a clinical (patient) sample device used in clinical research or diagnostics. The patient sample use is advantageous in a variety of situations, for example, where a permanent record of an assay result may be desired.

In preferred embodiments, the method also involves storing the sample device for an extended period of time, preferably without significant degradation of the labeled sample to generate a detectable light scattering signal. Such degradation can occur, for example, through bleaching, quenching, decay, or chemical degradation of the label, and/or through degradation the coating. Degradation of the coating can, for example, result in increased cloudiness or even opacity, increased coloration, and/or increased light scattering. In particular embodiments, the preserved sample device is stored for a period of at least 1, 2, 4, 6, 8, 10, 14, 21, or 28 days. In further embodiments, the preserved sample device is stored for at least one week, 1, 2, 4, 6, 8, 10, or 12 months, or even more.

The ability to preserve a sample device, and to store it as desired, without experimentally significant degradation of the detectable signal provides advantages in a variety of situations. For example, such preservation and the ability to store sample devices allows repeat reading of the assay results for an experiment, as well as delayed reading of assay results. This allows the sample device and/or assay results to be used across time and/or between different laboratories while still obtaining comparative results. Such comparative results can be obtained even when different instruments are used, by calibrating the instruments or results with a standard “calibration” sample device (e.g., a “calibration slide”).

As used herein, the term “sample device” refers to a physical item that is configured to retain a sample of some material, e.g., an analyte or a material that may contain an analyte. Preferably the sample device has a surface or surfaces on which the sample or samples are attached. The attachment may be direct or indirect. Non-limiting examples of sample devices include slides, chips, plates, microtiter plates, and membranes.

The term “forensic sample device” refers to a sample device that has a sample or samples relating to a law enforcement investigation and/or legal proceeding. Thus, for example, the forensic sample device can have sample(s) from a suspect(s) and/or victim(s), or can have crime scene samples.

The term “identification sample device” refers to a sample device with sample(s) selected to provide identification of an individual organism, preferably a mammal, more preferably a human. For example, the device may be an array providing genotyping information to distinguish the sample source individual from some or all other individuals.

The terms “clinical sample device” and “patient sample device” refer to a sample device with samples from one or more individuals selected for medically-related purposes (e.g., clinical or medical research purposes). The sample device and the associated samples are typically selected and configured to diagnose the presence, absence, or status of a disease or condition in the patient, or the susceptibility or resistance to the occurrence or certain courses of development or outcomes of a disease or condition. Alternatively, a patient sample device is configured for research purposes, for example, to provide a comparison of genetic characteristic or gene expression levels between a patient or patients having a disease or condition with one ore more control individuals not having the disease or condition and/or individuals having a different form or severity of the disease or condition.

The terms “clear”, “optically clear”, “transparent”, and “transparency” refer to the ability of a material, e.g., a coating material and/or support material, to pass light sufficiently and sufficiently free from cloudiness and the like that images are readily discernable through the material. In the case of materials that are in the light path for illumination or detection for a sample, the term indicates that, in the amounts used in a particular case, the material does not substantially interfere with the passage of light through the material to an extent to prevent reproducible repeat detection of scattered light from light scattering particle labels associated with the sample. Such interference may include, for example, absorption, reflection, and/or scattering by the material. Highly preferably, in the amounts used in the present invention, an optically clear material does not reduce the intensity of light passed through the material by more than 30, more preferably by no more than 20%, still more preferably by no more than 10%, and most preferably by no more than 5%, 4%, 3%, 2% or 1%. It is understood, however, that these terms do not necessarily mean that the material is completely colorless. However, the amount of color and/or the wavelengths of light not passing through the material are such that it does not prevent use of the coating in the assay. For example, even a relatively highly colored material may be used if the coating is sufficiently thin that the fraction of light reflected or absorbed is small enough to not preclude effectively carrying out the assay, and may be small enough to be negligible. Likewise, the wavelengths of light reflected or absorbed may be such that it does not prevent effective illumination and detection of the labels.

As used herein, the term “solidifying” refers to a transition from a liquid to a solid state, where the term “solid” has its common meaning, indicating that the material has sufficient coherence of form to distinguish from liquids and gases. At a minimum, the material has sufficient coherence of form that there is no fluid flow visible to the human eye when held in any position for 10 hr for amounts and shapes of a material as used in the present invention. Highly preferably the material shows no deformation visible to the human eye when subjected to moderate pressure with a human finger for 5 seconds. Solidifying may involve various processes, e.g., drying, cross-linking, polymerization, and/or other reactions that reduce the freedom of movement of component molecules in a solidified material sufficiently to result in a solid. Solidifying differs from a situation in which a suspension or colloid of solid particles in a liquid or gas are formed. In such suspensions or colloids, the bulk solvent remains liquid or gas and only the colloidal particles are solid material, while in the present solidified material the chemical and physical interactions resulting in the solid occur through the solidified coating and are not restricted to colloid particle scale.

As used herein, the term “solution” refers to a material with a predominantly liquid bulk property. Thus, the term includes true solutions, as well as suspensions, liquid medium colloids, and emulsions.

As used herein in connection with sample devices or other solid phase items, the term “chip” refers to a substantially planar solid substrate with surface area of 1 in² or less. Preferably the substrate is optically clear, e.g., glass or plastic although other material supports can be used.

As used in connection with sample devices or other solid phase items, the term “slide” refers to a generally planar solid substrate with a surface area greater than 1 in² up to 4 in² inclusive. Preferably the substrate is optically clear. Glass microscope slides with dimensions approximately 1 inch by 3 inches are an example. While slides with surfaces that are substantially uniformly planar are preferred, slides may have depressions, permanently attached or removable well structures, or other surface structures useful or not preventing use of the slide in the intended assay.

Likewise, the term “plate” refers to a solid substrate with a generally planar surface having an area greater than 4 in². The plate may be substantially uniformly planar, or may have depressions, attached well structures, or other structural features. In some embodiments, the plate has depressions, e.g., wells, for containing liquids, for example, microtiter plates (e.g., 96-well, 192-well, and 384-well plates). In other embodiments, a plate may have either permanently mounted or removable well structures affixed to the surface of the plate.

The term “chamber slide” refers to a slide that has a chambered well or wells on a surface for holding fluid samples during processing, e.g., during incubations. Typically the upper structure defining the well sides is made of polystyrene or the like, and is sealed to the slide surface with an elastomeric gasket, such as a silicon rubber gasket. The gasket and upper structure is generally removable. Thus, individual samples can be applied to different areas of the slide. Typically, but not necessarily, the well structure is removed prior to coating and/or reading the slide.

In connection with membranes and solid supports, the term “attached” refers to physical retention of the membrane by the support with sufficient strength to retain the membrane under normal handling in any position. This is distinguished from “supported”, which refers to retention of the membrane on the solid support under the force of gravity, but which may not retain the membrane in position in all orientations. Support does not involve physical bonding, clamping, or similar strong chemical or physical linkage. In contrast, the term “bonded” indicates that the membrane is attached to the solid support through the use of chemical bond interactions and/or an adhesive.

In the context of this invention, “membrane” refers to a thin, flexible impermeable or microporous material, preferably synthetic material. Preferably pores or channels in the membrane are no larger than 20 μm, more preferably no larger than 10, 5, 2, 1, 0.5, 0.2 or 0.1 μm, or in a range specified by any two of these specified endpoints. A membrane may be, for example, a uniform sheet of material with essentially uniform composition, e.g., a film, or a fibrous material, e.g., woven or matted fibrous material. Examples of commonly used materials include nylon, nitrocellulose, polyvinylidene fluoride (PVDF), and cellulose. The membrane can have any of a range of surface areas, with the choice typically determined by the intended application, e.g., the size and number of features in an array. Thus, in particular embodiments, the membrane sample device has an area of less than 1 in², 2 in², 4 in², or 10 in², though larger membranes can also be used.

The term “dark conditions” refers to dim light as perceived by humans with normal vision, but, unless otherwise specified, does not require complete dark unless clearly specified. Recognizing that UV light is particularly significant for degradation of materials due to photo-damage and UV-induced chemical changes, dark conditions involve reduction of ultraviolet light in particular to an intensity no greater than 10% the intensity produced by a standard 40 watt fluorescent light bulb designed for work or residential area illumination measured at a distance of 2 meters and averaged across the UV spectrum. More preferably, the dark conditions UV intensity is no more than 5%, 2%, 1%, 0.5%, 0.2%, 0.1%, or even less as compared to the fluorescent light bulb intensity as indicated. Likewise, preferably other wavelengths are reduced to the same intensity % range as the UV. Such dark conditions exclude brief periods when a storage container or other space may be opened, e.g., for introduction or removal of a sample device.

In a related aspect, the invention also provides a preserved sample device, which includes a solid phase medium with light scattering particle labels attached, and an optically clear solid coating covering the light scattering particle labels. In most cases, the light scattering particles labels are attached, directly or indirectly, to analytes on the solid phase medium.

In preferred embodiments, the sample device includes a solid phase array. Likewise, in preferred embodiments, the sample device includes a slide, a chamber slide, a microtiter plate, an array chip, a membrane, or the like.

In another aspect, the invention provides a one-step method for transparifying and preserving a sample membrane, by treating the membrane with a solidifying, non-dissolving, optically clear solution. Preferably the sample membrane has light scattering particle labels attached to it.

In preferred embodiments, the sample membrane is associated with an optically clear solid phase support. Preferably the solid phase support is glass or plastic.

In preferred embodiments, the sample membrane is attached to, supported by, and/or bonded to the support. For example, in particular embodiments, the membrane is attached to a frame, supported by a slide, or bonded to a slide.

In embodiments where a membrane is bonded to a slide or other support, preferably the bonding uses an adhesive. The adhesive may be in various forms, for example, sheet, liquid, and semi-liquid. Preferably, but not necessarily, the adhesive is optically clear following bonding. Such optical clarity is especially useful when illumination and detection are on opposite sides of the support, but can also be beneficial in other configurations, e.g., to reduce non-specific scattered light. In other embodiments, the bonding involves direct chemical interaction between the membrane and the support, e.g., a functionalized surface of the support.

As used in connection with membranes and solid phase supports, the term “associated with” refers to any manner of interaction that retains a membrane adjacent to the solid phase support by interaction between the membrane and device. Thus, the term includes, for example, attached to, resting on, bonded to, clipped to, and supported by the solid phase support.

As used herein in connection with membranes, the term “transparifying” refers to substantially reducing the light scattered from the membrane under particular illumination conditions, e.g., by contacting the membrane with a fluid that reduces light scatter from the membrane. Typically and preferably the process increases the transparency of the membrane.

Preferably the fluid is an optically clear fluid.

The term “non-dissolving” indicates that the solution does not dissolve the membrane matrix, i.e., leaves the membrane structure substantially intact.

In another aspect, the invention provides a method for reducing background light scattering in an analyte assay utilizing light scattering particle labels, by coating at least a portion of a sample device having attached light scattering particle labels with a solidifying, optically clear solution.

As with embodiments of aspects above, in preferred embodiments, the sample device includes a solid phase array, a slide, a chamber slide, an array chip, a microtiter plate, or a membrane.

In yet another aspect, the invention provides a method for enhancing specific detection of light scattering particle labels in an analyte assay, by coating at least a portion of a sample device having attached light scattering particle labels with an optically clear, solidifying solution, where the solid coating resulting from the coating provides refractive index enhancement for the scattered light signal from the particles.

In particular embodiments, the method involves a sample device and/or storage as in the first aspect above. Also in particular embodiments, the coating material is one described herein.

The phrase “enhancing specific detection” and phrases and terms of like import refer to improving the ability of a detection system to distinguish between background and a specific signal. In the context of analyte detection systems, the specific signal is signal associated with the specific analyte. Such enhancement can involve relative or absolute reduction in background signal and/or relative or absolute increase in specific signal.

In still another aspect, the invention provides a method for delayed detection of analyte on a sample device having attached light scattering particle labels bound with analyte. The sample device has an optically clear solid coating. The method involves detecting light scattered from the labels as an indication of the presence or amount or both of at least one analyte on the sample device, following storage of the coated sample device for a period of at least one day, preferably at least one week. In this method, the light scattered from the labels under the same illumination and detection conditions is stable over the period for the same illumination conditions.

In order to provide the coated sample device, in preferred embodiments, the method also includes coating at least a portion of the sample device with an optically clear solidifying solution prior to the storage, and/or storing the solid coated sample device.

While this method can be used simply for delayed initial detection, in preferred embodiments, the light scattered from the labels is also detected as an indication of the presence or amount or both of at least one analyte on the sample device, prior to storing the device, or at least before storing the device for a period greater than a few hours, e.g., greater than 1, 2, 4, 6, 8, 12, 16, 20, or 24 hours.

Indeed, in preferred embodiments, the storing and detecting are performed a plurality of times.

The period of storage can vary, with the limit on reproducible repeat detection generally limited by the stability of the coating material selected, in view of the storage conditions selected. Parameters that can significantly affect the practical storage period include extent of exposure of the coating to light (especially ultraviolet light), storage temperature, exposure of the coating to chemicals that can chemically react with the coating material at a significant rate. In preferred embodiments, a storage period is at least one week, 2 weeks, one month, 2 months, 4 months, 6 months, 9 months, one year, or even longer.

Highly preferably the light scattered from the particles remains substantially constant (under the same illumination and detection conditions) following the storing.

Also in preferred embodiments, the method also includes washing the coated sample device before initial and/or repeat detection. Such washing is useful to remove background light scattering, e.g., from dust particles. The coating protects the light scattering particles from being washed or abraded away.

Preferably the wash conditions are physically and chemically mild. Thus, for example, preferably there is no abrasive cleaning, and the wash solution(s) are chemically mild for the particular coating. Preferably the wash solution is an aqueous solution. Such aqueous solution may contain a buffer(s) and/or mild detergent and/or low to moderate ion concentration. Other or alternate compatible components may also be present. Other solvent compatible with the coating may be used instead of water. A compatible solvent (or solution) does not significantly degrade the coating in a manner interfering with repeat or delayed detection. In some cases, a solvent or solution (and accompanying wash conditions) may be selected that dissolves a thin layer of the coating, thereby providing a fresh coating surface. Preferably such dissolved thin layer does not exceed 1, 2, 5, 10, or 20% of the coating thickness.

Alternatively, or in addition, the sample device can be re-coated using the same or a chemically compatible different optically clear solidifying solution. Upon hardening of the coating, the sample device can be reanalyzed. This approach is useful in a variety of situations, for example, where there is accidental scratching or dust accumulation due to improper storage and handling.

In yet another aspect, the invention provides an assay method for detecting the presence or amount or both of an analyte on a sample device having light scattering particle labels bound with analyte attached thereto. The method involves illuminating the light scattering particle labels with light, and detecting light scattered from the labels as an indication of the presence or amount or both of one or more analytes that are present on the sample device.

In preferred embodiments, the sample device includes a solid phase array, a slide, an array chip, a microtiter plate, or a membrane. The assay may be performed in a variety of ways, for example, as described in Yguerabide et al., U.S. Pat. No. 6,214,650 and WO 99/20789.

In particular embodiments, the sample device is preserved, detection is delayed, a solid coating provides refractive index enhancement, and an assay is a repeat assay following a period of storage of a solid coated sample device.

In a related aspect, the invention provides a kit. The kit is suitable for carrying out the aspects described above, e.g., for performing assays, preserving sample devices, and the like, as well as other similar uses. The kit includes a volume of an optically clear solidifying solution and a quantity of analyte-binding light scattering particle labels.

Typically the kit will be packaged in a single container. The optically clear solidifying solution is highly preferably packaged under conditions such that the solution will not solidify for a period of at least one week, more preferably at least one month, still more preferably at least two months, and most preferably at least 6 months.

The light scattering particle labels can be supplied in the kit in various forms, depending on the intended application, e.g., for use directly with assays, or for use in constructing custom assays. Thus, in certain embodiments, the light scattering particle labels have a moiety or moieties that bind to analyte under binding conditions. Such moieties include without limitation, specific oligonucleotides, antibodies and antibody fragments, specific antigens, haptens, biotin, aviden and streptaviden, as well as other members of specific binding pairs and other molecules that provide specific binding. The binding to an analyte can be direct or indirect. Likewise in certain embodiments, the light scattering particle labels have moieties that bind to analyte binding molecules under binding conditions. For example, the particle can have on its surface a moiety for attaching a nucleic acid or a protein, or other molecule that can provide direct or indirect analyte binding.

The kit can also include at least one sample device, e.g., at least 1, 2, 4, 6, 8, 10, or more sample devices. As with aspects described above, such sample devices include without limitation arrays, microarrays, array chips, slides, microtiter plates, and membranes.

As indicated above, use of calibration slides or other calibration sample devices is beneficial, e.g., to assist in cross-instrument, cross-experiment, and/or cross-laboratory comparisons of assay results.

Therefore, in another aspect, the present methods for archiving various sample devices are also used for the purpose of establishing calibration samples with RLS particles. As described above, RLS particles are stable labels from the standpoint that the light scattering signal obtained is not subject to decay, bleaching or quenching. Thus, the method for preparing calibration sample devices involves depositing predetermined quantities (or ratios of quantities) of RLS particles in or on a sample device having the desired format and coating at least a portion of the device with an optically clear archiving agent, highly preferably an optically clear solidifying solution. Preferably different quantities or dilutions of RLS particles are deposited at a plurality of respective spatially discrete sites in or on the device.

For example, array (including microarray) calibration devices can be prepared by printing RLS particle dilutions on an array. The array is then coated or otherwise archived. After archiving, this calibration slide can be used to adjust or calibrate the corresponding light scattering signals across different detection instrument units and/or across different experiments or determinations with the same instrument. The use of such reproducible calibration sample devices therefore allows more direct comparison of experimental results obtained in different laboratories, with a higher level of confidence.

A variety of different RLS particles may be used, and a single calibration device may have one or more different types of particles. Preferably, the type of RLS particles on a calibration sample device includes the type or types present on a sample device with which the calibration sample device is used. Examples of RLS particles that can be used include generally spherical gold, silver, and combined gold and silver particles of 20, 40, 60, 80, 100, and 120 nm diameter.

In a related aspect, the invention includes a method for providing reliable comparison of assay results between different experiments by calibrating an assay apparatus that detects scattered light signals from RLS particles with a calibration sample device, thereby providing a first set of assay results normalized or standardized relative to a calibration standard, in a separate experiment calibrating an assay apparatus as specified with a calibration sample device, thereby providing a second set of assay results normalized or standardized to a calibration standard. Similar calibration and assays can be performed providing a third or more sets of assay results. Calibration of the different assay experiments to a calibration standard allows reliable comparison of the results between the different experiments. The calibration standards for the different experiments may be the same or may have a known conversion or scaling factor, curve, or equation. The results from at least two of the different experiments are compared, with the results scaled such that the same sample produces equivalent assay results in the different experiments.

In an exemplary embodiment, a calibration sample device is used to construct a standard curve (e.g., based on one or more particle dilution series on the calibration sample device) in an analyte assay using RLS particle labeled analyte. Either the same or different calibration sample device is used in different experiment, with the same or different analyzer in the same or different laboratory. Where different calibration sample devices are used, the different devices have a calibration factor associated with the device that allows comparison with the other calibration sample device or devices.

In another related aspect, the invention concerns a calibration sample device, e.g., a slide. The device has different amount of particular types of light scattering label particles attached at different locations on the device, and is at least partially coated with an optically clear coating, highly preferably a coating solidified from an optically clear solidifying solution.

In particular embodiments the calibration device has at least one dilution series of particles, e.g., a series of 2-fold, 5-fold, or 10-fold dilutions; has a plurality of different types of particles; and/or is packaged with a data sheet providing calibration data for the calibration device. (alternatively, such calibration data can be written in one form or another on the device itself.

A number of different coating materials can be used in the present invention. Those of ordinary skill in the art will readily be able to select a preferred material for a particular implementation. Examples of candidate materials include a variety of polymer materials, such as lacquer, varnish, polyurethane, acrylic, polyester, carbohydrate polymers, epoxide polymers, and organic-inorganic network materials. Coating materials can also include co-polymers of different components. Exemplary commercial products are available under the names Rustoleum® (clear coat paint), Krylon® (clear coat paint), Deft® lacquer, Plascron® and Break-Through® from Midwest Industrial Coatings, Inc., and Ficoll® from Sigma-Aldrich, among others. Numerous other products that can be readily tested for suitability are available and additional products are being developed and can be tested. Examples also include biopolymers and other water-soluble materials that cure or dry to form an optically clear coating. These materials may be advantageous in view of the manufacturing, shipping and handling issues associated with many organic-based coatings.

As described above in connection with the first aspect, while optically clear solidifying solutions can be used, in other embodiments of the various aspects optically clear non-solidifying solutions can likewise be used, e.g., in the manner described above.

In the various aspects and embodiments for which specific values are provided, unless clearly indicated to the contrary or the context indicates only discrete exact number, e.g., integers, are suitable, those numbers can be the specified value plus or minus 20%. This variation also includes cases where the covered variation is plus or minus 10%, 5%, 2%, or 1%, as well as all other integer values between 0 and 20%.

Additional features and embodiments will be apparent from the following Detailed Description and from the claims.

### BRIEF DESCRIPTION OF THE DRAWINGS

FIG. 1 is a bar graph showing exemplary signal to background ratios for several coating materials on glass slides with 80 nm gold RLS particles.

FIG. 2 is a microarray layout used for illustrating the membrane transparifying and archiving method.

FIG. 3 is a bar graph showing exemplary signal to background ratios for 3 lacquer solutions used as coating materials on nitrocellulose membrane with 80 nm gold RLS particles. The identifier, d100 refers to 100% Deft® lacquer. D50egme50 refers to a solution of 50% Deft lacquer and 50% 2-butoxyethanol. P50egme50 refers to a solution of 50% Parks lacquer and 50% 2-butoxyethanol.

### DETAILED DESCRIPTION OF THE PREFERRED EMBODIMENTS

The invention described herein relates to compositions of matter, formulations, and processes useful for fixing the location of analyte labels to a solid surface in substantially irreversible manner. The present invention can be applied to any sample device for which it is desired to immobilize detectable label, especially photodetectable label. The description herein is presented with emphasis on the use of resonance light scattering (RLS) particles, however, the invention is not so limited. Examples of other types of labels include fluorescent labels, luminescent labels, chromogenic labels, and radioactive labels, among others. These compositions and methods are designed to immobilize and protect attached label, to maximize signal intensity from RLS particles, and/or to minimize non-specific background scattering, highly preferably in a physical form that is durable and convenient to handle. As indicated, while the present invention is particularly advantageous for use with RLS technology, the materials and methods described herein can also be applied to other types of labeled samples, e.g., for reduction of background light scattering and/or preservation of labeled sample on a sample device. As an example, the invention may be applied to fluorescently labeled samples.

Resonance light scattering provides a highly sensitive method for detecting the presence of submicroscopic particles. This technology preferably uses gold and/or silver particles of uniform size, typically in the range of 40-120 nm in diameter, though particles in a greater range can also be used, e.g., 1-500 nm, or 20-200 nm, or 30-300 nm. When illuminated with white or other polychromatic light under appropriate conditions, these particles scatter light of a specific color and intensity, with very high efficiency. The particles can be derivatized with a variety of biomolecules to allow specific particle binding for detection and potentially quantitation of many different target moieties, for example, specific haptens, antigens, proteins, peptides, carbohydrates, lipids, small molecule ligands, nucleic acids, and the like. RLS detection systems also provide excellent spatial resolution for applications requiring precise microscopic localization.

In order to reduce the length of this description, discussion of methods and apparatus for use in RLS methods is not written out in detail herein. Such description is known to those of ordinary skill in the art, and is available, for example, in the Yguerabide references referred to and incorporated by reference above. Likewise, methods using other types of labels are also not written out in detail herein. Such methods are described in many documents, and are well-known to those of ordinary skill in the art.

Use of the present invention can provide a number of advantages in particular applications. As described below, these can include but are not limited to one or more of the following: reduction of background signal, enhancement of light scattering efficiency, sample protection, and enabling consistent repeat analysis.

One practical factor encountered in applying RLS technology on solid surfaces or membranes is that dust, particulate contaminants, surface irregularities or optical properties of the underlying substrate that scatter light will contribute to a non-specific background signal. That background signal may obscure the primary scattering signal from the label particles. Also, the scattering efficiency of the particles being used for RLS detection depends on the refractive index of the medium surrounding them, with a higher refractive index medium (for example, water) giving a stronger signal than a lower refractive index one (for example, air).

Thus, it is preferable to use a medium with a refractive index that both enhances RLS particle scattering, and suppresses non-specific background scattering. Liquids that have these properties have been described (e.g., Yguerabide & Yguerabide, 1998, supra.) However, media that remain liquid tend to be messy to use and to be susceptible to evaporation and contamination. As a result, a solution that can be hardened to form an impermeable surface can be preferable to a liquid in many applications.

In addition to background reduction and/or specific light scattering enhancement (or in the alternative) a solid coating can provide physical and/or chemical protection for labeled samples. In this respect, the ability to achieve precise spatial localization with RLS is particularly useful for cell biology, molecular biology, and analytical chemistry applications in which the target to be detected is immobilized on a solid surface, for example tissues, whole cells, sub-cellular components, or manufactured microarray systems. If the binding of the particles to their targets or to the surface is accomplished via a chemical reaction or surface adherence, it is susceptible to reversal. Covering bound particles with a solid surface, impermeable to damaging liquids or physical forces that might dislodge the particles from their original location eliminates this problem.

Further, materials analyzed using RLS detection can potentially be re-analyzed a large number of time (potentially effectively infinite), providing essentially the same quantitative output of scattered light each time (with the same illumination conditions). This is because the RLS signal does not quench, fade, decay, or bleach, as does fluorescence, chemiluminescence, radioisotopes and many chromogenic detection systems collectively representing different labeling technologies. It is therefore possible to construct quantitative RLS calibration standards, enabling normalization of results obtained by different operators, at different times, with different equipment, to obtain absolute quantitative results. This kind of universal calibration and absolute quantitation is not currently possible using fluorescence or other detection reagents or equipment, where only relative signals can be obtained. Physical durability, for example, by coating with a solid surface, is an important property to ensure the stability of these calibration standards over time.

Coating Process

Coating of sample devices can be performed in a variety of ways, including without limitation spraying, dipping, and pouring methods. One of ordinary skill in the art of applying coatings will recognize that selection of a suitable coating method will depend on the specific coating selected, the character of the resulting finished coating needed, convenience, cost, and other process factors. Thus, the best application method can differ in various situations.

As is commonly understood in the field of application of thin coatings, spraying may be airless, involving atomization of the fluid as it flows under high pressure from a spray nozzle. Other spray systems utilize a stream of gas (usually air) under pressures of about 30-80 psi to propel and atomize the coating fluid. Spray application may be suitable where the flow characteristics of the coating after application allow formation of a sufficiently smooth and defect free surface to avoid difficulties with light scattering from surface imperfections. Additionally, spray methods are more likely to be suitable in cases where overspray is not a significant problem, and thus is more likely to be applied in cases where large areas are to be coated at the same time.

Dipping typically involves dipping a sample device in a volume of coating material sufficient to immerse at least the portion of the device surface having attached label or that is otherwise desired to coat. Typically the device is allowed to drain for a period of time to remove excess fluid coating before the coating solidifies. The device may be allowed to harden in a vertical or inclined draining position, or may be placed in a generally horizontal position to minimize strain and irregularities in the coating as it solidifies. Spinning, e.g., in a low-speed centrifuge can also be used to remove excess coating solution.

Pouring typically involves placing a sample device in a generally horizontal position and pouring the coating material on the upper horizontal surface. The device may remain in the horizontal position while the coating solidifies, or may be inclined to facilitate draining. As indicated in connection with dipping, spinning can also be used.

Persons familiar with coating materials and processes will recognize many different variations in coating methods that can be used appropriately with specific coating materials.

Coating properties

Low background scattering

Any of a number of different types of coating materials can be used in the present invention. Consistent with the description above, the specific material selected for a particular application will depend on the properties required for that application. In addition, the properties of an available material may be modified to provide a more advantageous material. Examples of materials that may be appropriate for particular situations include without limitation, coatings of polyurethanes, polyesters, acrylics, lacquers, epoxide polymers, carbohydrate or other bio-polymers, as well as chemically and optically compatible combinations and copolymers.

In general, it is beneficial to select a material that provides an optically clear coating with low non-specific light scatter. Such non-specific light scatter can arise, for example, from inhomogeneities in the material, including, for example, contaminant particulate matter, solidified material with different refractive index, and bubbles. As a result, a coating material is preferably selected that does not contribute significant background scatter. Further, the handling of the material and the coating process should be done to minimize introduction of scattering materials. Thus, for example, the material should be protected from dust and other airborne particles, and handled in a manner to avoid creation of bubbles. However, if particles or bubbles are present, such can generally be removed by filtration and de-gassing respectively.

Following initial coating, the coated sample device should be handled in a manner to avoid introduction of non-specific light scatter. In general, it is beneficial to have a surface on the solidified coating that is as free as possible from defects. Such defects can include foreign material and/or surface irregularities. For example, during solidification, the coating should be protected from particles that could deposit on the coating surface. Likewise, the solidification should be carried out in a manner that does not introduce surface irregularities, e.g., contacting the surface before the coating is fully solid or permitting flow of partially solidified material. In this regard, agents that are fairly nonviscous and exhibit self-leveling properties are particularly useful.

Clarity

Coatings useful in this invention should allow for light transmission in a largely unobstructed, non-scattering manner. Thus, opaque coatings generally cannot be used. In addition, particularly for use with RLS labels, as indicated above, the coating should not contribute significantly to background light scattering. Thus, translucent coatings are not preferred, even though they permit the passage of substantial light. It is highly preferred that the coating lack any visible cloudiness or similar characteristics.

However, it is only important that the coating is transparent with respect to relevant wavelengths of light. For example, in particular applications, a coating may highly absorb ultraviolet, or near ultraviolet wavelengths without interfering with performance of an assay, due to the light wavelengths detected. Similarly, a material may significantly absorb infrared wavelenghts, but still not interfere with performance of an assay. Preferably, the coating should not prevent use of visible wavelengths of light, especially in the 400 to 700 nm wavelength range, or at least 450-700 nm range.

Durability—chemical & physical

In many applications, it is highly beneficial if the coating is physically and/or chemically durable. If a sample device is to be read immediately and not stored for later reading, these characteristics are of less importance, a softer and/or less chemically resistant coating may well be acceptable. However, in general, a hard coating is preferred. Resistance to chemicals that may be encountered is also advantageous.

With respect to chemical resistance, in particular embodiments the optical properties of the coating are unaffected by a brief rinse with water and preferably are unaffected by exposure to water at room temperature for up to 1 hour, preferably up to one day, or longer. Preferably the coating is also similarly resistant to solutions with which a coated sample device is likely to come in contact, for example, one or more of the following: common buffers used in biological laboratory practice, microscope immersion oil, detergent solutions, ethanol, propanol, and the like, as well as mixtures of ethanol and/or propanol and water.

With respect to physical durability, the most important characteristics are scratch resistance and resistance to embedding of foreign particles. While the Moh's scale is usually used in connection with minerals, applying it to coatings, a coating for use in this invention preferably is at least 1.5 more preferably at least 2, 2.5, 3 or 3.5 on that scale, with higher values being more preferred. In terms of exemplary comparisons, preferably a solidified coating has a hardness and scratch resistance greater than the average for commercial outdoor application alkyd enamel paints applied according to manufacturer recommendations and allowed to dry for one week at 23 degrees C. with 50% humidity.

Thickness

Suitable coatings for the present invention can be any within a range of thickness. What is important in performance of an assay is that the coating allow effective illumination and detection. For sample devices having small features and for detection of single particles, it is highly preferred that the coating not distort the signal image to an extent that degrades resolution below the level needed in a particular application, e.g., to be able to distinguish adjacent microarray features. Typical coating thicknesses will be in the range of 1 micrometer to 1 mm inclusive, preferably in the range 1 micrometer to 0.1 mm, or 0.02 mm to 0.1 mm.

Viscosity Modification

While any of a number of suitable coating materials can be used, modifying the viscosity of coating materials employed herein can be beneficial.

It has been observed that dilution of polyurethanes, lacquers and other clear coat finishes with highly volatile ketone-based solvents has the effect of reducing liquid viscosity, and reducing cure time of the original materials. These features can provide the following benefits:

1. User handling time is decreased (e.g., 3-4 hour cure time on original form, ˜1 hr on the diluted form).

2. The resulting tegument is thinner (viscosity reduction allows more complete run-off).

3. Background levels are reduced (concentrated form has higher levels of particulate).

4. No lip is apparent on the slide after drying.

This approach to altering flow character and cure time of paints, lacquers, and polyurethanes is used among specialty paint and hardware stores and in application of optical clear coatings, paints, protective barriers etc.

The converse may also at times be desirable. That is, decreasing volatility of clear coat liquids prior to application can be beneficial in certain circumstances. For example, addition of 2-butoxyethanol, as well as, some classes of aromatic compounds (e.g. compounds such as benzaldehyde and toluene that contain an aromatic ring, generally a hydrocarbyl ring, most often a phenyl ring) have the effect of increasing the cure time by decreasing the overall clear-coat solvent volatility. This effect of increased cure time is desirable, for example, in the case where rapid cure times may introduce frost upon the coating; a phenomenon attributed to moisture deposition upon the coating surface during the cure process.

The addition of these solvent or thinning reagents may also as a consequence improve the flow character of the coating. In rapid curing clear-coats, the flow of liquid is rapidly frozen to the slide, which may introduce striations on the coating surface. The result is an imperfect surface, potentially introducing additional light scatter.

The same approaches may also be applied to other coating materials using chemically compatible higher or lower volatility solvents. Such compatible solvents can readily be selected based on the known chemistry of a particular coating and/or by empirically testing or confirming compatibility.

Membranes

Membranes bound to solid supports present a formidable obstacle to RLS technology. Optical scanning methods for fluorescence detection, such as those utilized in confocal microscopy, may be put to good use on solid supports not rendered optically clear. The situation can differ for resonance light scattering detection. Incident white light can be scattered by unclear substrates, non-specific particulates, molecules and substrate surface irregularity. Particularly relevant to membranes bound to solid supports is the lack of substrate clarity.

Solid support bound membranes are a substrate for deposited molecules relevant to biotechnology. These membranes should be rendered optically clear to obtain a robust and specific signal from bound, immobilized RLS particles. In addition, as described above, it is advantageous to coat the membrane to provide protection, preservation, and/or signal enhancement. The present inventors discovered a method and a class of reagents which simultaneously transparifies cellulose nitrate membranes while producing a durable tegument around the solid support. In addition to cellulose nitrate membranes, this technology can be applied to many of the membranes used in biotechnology, such as nylon and polyvinyl difluoride (PVDF).

The use of liquid materials to clarify membranes has been practiced. Such clarifying is described, for example, in Brooks, U.S. Pat. No. 6,165,798, which is incorporated by reference herein in its entirety. The Brooks patent mentions the use of polyvinlypyrrolidone (PVP), polyethyleimine (PEI), and PEI+ water in addition other agents that dissolve the membrane. Other liquid clarifying materials include Type A immersion oil and benzenemethanol (refractive index=1.539). The drawback to these approaches is that they are neither “user friendly”, nor compatible with routine instrument operation due to their inherent need for “wet” chemistry.

While refractive index matching materials can achieve membrane transparency, so too can solvents/solutions with lower refractive index through the assistance of chemical modification acting to substantially reduce cross-linking in a cross-linked membrane polymer. The observation that 100% ethanol can render a nitrocellulose membrane nearly transparent indicates that simple reduction or modification of cross-linking structure can facilitate transparification. Membrane chemical modification by the transparifying agents after the bioassay has been completed is of little consequence. In addition, disassembly of the membrane's extensive architecture may help to reduce the residual haze produced in extensively cross-linked polymer networks. This residual haze can easily be visualized with the aid of a Tyndall Beam. Thus, in certain embodiments, the coating material also includes an agent or agents that chemically modify the membrane, e.g., by reducing crosslinking in the membrane, though without the extensive dissolution described in Brooks et al., U.S. Pat. No. 6,165,798.

There are substantial benefits of a reagent capable of both transparification and archival of membranes bound to solid supports with a single treatment. These include, without limitation:

1. Membrane transparification minimizes non-specific scatter introduced by the substrate on which the immobilized particles have been attached.

2. The archival process has the end effect of preserving the specifically attached RLS particles in a quasi-liquid medium, thereby enhancing RLS particle light scattering intensity. Properties inherent to liquids yield greater RLS particle signal intensities, relative to air.

3. The archival process is capable of dissolving and transparifying much of the non-specific scattering debris inseparable from the solid support by routine processing.

4. The archiving results in a smooth, regular surface.

5. The archival process both protects and preserves the membrane, as well the specific signal retained on the membrane, indefinitely. RLS particles are not subject to compromised signal strength over time. The marriage of this quality with archiving lends a tremendous advantage over other light detection technologies in the frequency and duration over which an RLS particle signal can be read.

6. The solid support can be cleansed with mild solvents anytime after fully curing the archiving/transparifying agent to remove unwanted accumulated debris and oil.

Storage

For sample devices that are to be stored for later analysis (initial or repeat), it is highly preferable to store the device in a manner that avoids creation of defects that can degrade the analysis. Such defects can be created in various ways, for example, photo damage, physical damage, chemical damage, and foreign material (e.g., dust) on the surface.

Many types of coating materials will be subject to photo damage. Such damage is especially likely to be created due to ultraviolet (UV) light due to the high energy of such light. Such photo damage can include introduction of color and physical degradation of the coating, especially the surface, with concomitant increase in background light scattering and reduction in reproducibility of illumination of the labels and detection of the specific signal.

Such photo damage can be reduced to low levels by storing the coated sample device in dark conditions. While the coating can be subject to photo damage when removed from the dark conditions, generally such damage will be negligible. Preferably the dark conditions include measures to reduce UV exposure as much as possible, but also preferably include reduction in exposure to other wavelengths. Conventional methods for dark storage conditions can be used, e.g., use of light blocking containers or storage in a dark room.

In addition to photo damage, coated slides can be subjected to physical damage. That is, the coating can be damaged by physical contact, thereby creating surface defects that can contribute to increase in non-specific background and/or reduced lifetime for the coating. Such physical damage can include, for example, abrasions, cuts, and embedded particles. Moderate care in handling will avoid most such damage, e.g., handling sample devices by the edges, avoiding contacting the surface with sharp or abrasive surfaces, and using care in cleaning dust or other particles from the surface.

Additionally, the coating surface may be damaged by chemicals. Such chemicals, may, for example, be in wash solutions and/or fumes. In many laboratory settings, fumes from a variety of different chemicals may be present. Depending on the chemical characteristics of the coating, the fumes may react with the coating, damaging the surface. Thus, in general, it is desirable to avoid contact with such fumes that will react with a particular coating, especially for extended periods of time. Likewise, if they are to be used, wash solutions should be selected that do not significantly react with a particular coating, either by chemically modifying the coating, or by dissolving the coating. (However, a slight dissolution can be advantageous as it can provide a new surface, removing or reducing slight surface defects.)

It is desirable to minimize deposition of foreign materials such as dust on a coating during storage or analysis. However, in the event dust or other materials are found on the surface, the solidified coating can be washed and/or cleaned with a gas stream (e.g., air or nitrogen). Such wash solution and/or gas should itself be essentially free of foreign materials that would deposit on the coating surface. In addition, as indicated above, the wash solution and/or gas should be selected that are chemically compatible with the coating material. Further, the surface cleaning should be conducted in a manner to avoid physical damage. For example, washing should be done to avoid abrasion damage to the surface, e.g., by using a gentle to moderate liquid stream without wiping or scrubbing. Physical damage can also be avoided by selection of a hard coating in preference to a softer coating.

In addition, in some cases, samples that have been archived and experienced physical damage due to surface scratches or other defects or contamination, the sample can often be recovered to its original quality by simply retreating the sample with the same archiving agent, or a different, chemically compatible archiving agent. This aspect adds to the permanency of the sample preservation using the present invention.

One of ordinary skill in the art will be familiar with the factors relevant to avoiding coating damage, Sensitivity or resistance of a specific coating to damage from a particular condition can also be determined empirically by exposure and inspection, e.g., under high magnification and/or in assay or assay simulating conditions.

### EXAMPLES

### Example 1

### Evaluation of Coating Materials

Light scatter is an area of concern; both the liquid, and especially the solid state of the candidate “Archiving” material should be free or maintain only the lowest levels of light scatter. A coating which introduces light scatter will increase background noise and reduce sensitivity of light detection. The solidified material is preferably colorless. However, this may not be critical as the coating layer is quite thin and the contribution of color minimal. The curing method preferably does not involve extraordinary manipulations, or, equipment. Curing times should allow enough time for physical handling after coating application but not require more than 6 hours. 1 Hour is an example of a reasonable cure time. Signal strength before and after coating for a number of candidate coating materials was tested.

A number of candidate archiving (coating) materials were tested. The results show that several suitable coating materials have been identified, which fulfill the criteria of ease of application (sheeting, viscosity), dry time, refractive index, optical clarity, hardness/scratch resistance, stability of raw material, solvent compatibility, and cost.

Tests were performed with microarray slides printed with a solution containing bare gold 80 nm particles using an automated microarray printing system (Cartesian Technologies, Irvine, Calif.) and quill pens (Telechem International, Inc., Sunnyvale, Calif.). A complete description of microarray technology including printing, slide processing and fluorescent detection can be found in Microarray Biochip Technology, Ed. Mark Schena, Eaton Publishing, Natick, Mass., 2000. The pattern printed was of 5 replicates (row)/metacolumn. The particles were diluted from 50 O.D. by ½ over 8 samples (columns). Two Metarows containing 4 metacolumns each were printed on the slides. The slides were then treated with several washes in biological buffers containing one or all of the following.

3×or less SSC

0.1% w/v SDS

0.2% w/v BSA

Casein

10 mM PBS

Purified Water

Forced Nitrogen Air for Drying

These conditions were adopted to approximate microarray manipulation during experimental processing. The actual conditions used in most experimental processes may be more rigorous and may deposit higher levels of scattering impurities on a processed microarray slide.

Images of microarray features before and after coating with candidate archiving materials were processed identically using a commercially available microarray image analysis program (ArrayVision, Imaging Research, Inc., Ontario, Canada). This means display ranges were matched, in addition to scanning exposure times on the instrument taking the measurement. All image data were collected using the ArrayWorXs automated microarray processing system (Applied Precision, Issaquah, Wash.).

Exemplary candidate Archiving materials tested included:

Fcll=Ficoll® 50%

Kryln=Krylon® Clear Coat Acrylic

Rstlm=Rustoleum® Clear Coat Paint

PolyU=Polyurethane

PR=Combination (1:1) Plastic (Craftics®) and Rustoleum® Clear Coat Paint

PVA=PolyVinylAlcohol—viscoelastic polymer.

All slides were prepared (archived) by dipping, with drying/curing at standard temperature pressure. Scans were taken before and after coating the slides. Scans taken before coating were of the slides in the cleanest state that can be achieved after spotting (i.e. there was no further processing of the slides after spotting). It should be noted that treatment with biological buffers has typically shown a significant introduction of background noise as a result of trapped salts, proteins, small molecules and particle contaminants.

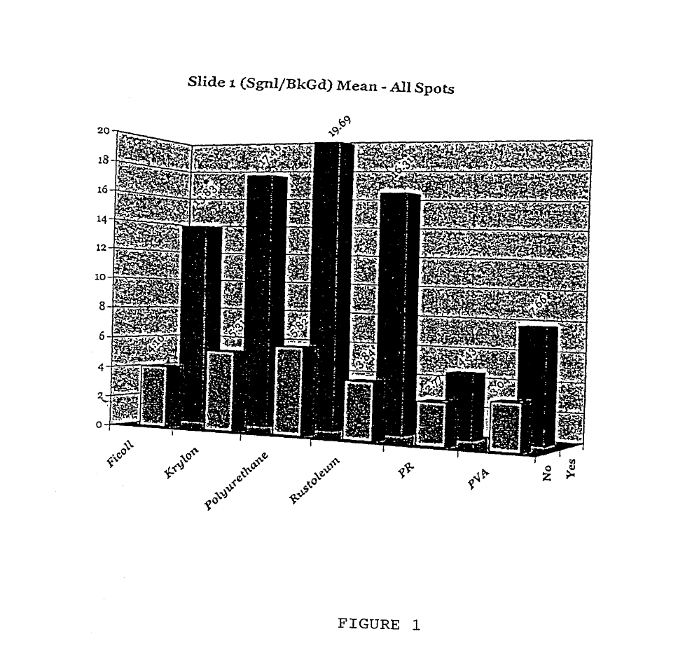

No loss in signal is observed, but a significant increase is observed in signal to background averages across all microarray features. FIG. 1 is a graph showing representative average signal to noise ratios for exemplary coatings. As shown, there was a dramatic increase in signal to background averages across all spots on “Archived” slides. In some of the better performing coatings, signal to background averages increase approximately 4-fold relative to uncoated slides.

### Example 2

### Transparifying and Archiving Membranes

This example describes the production of nitrocellulose membrane bound glass slides for the specific application of RLS particles. Further described is a process of nitrocellulose membrane transparification and archiving, using a solution that both clarifies the membrane and hardens to protect the membrane. The results show that several candidates for membrane transparification and archiving solutions have been identified which fulfill the criteria of ease of application (sheeting, viscosity), dry time, refractive index, optical clarity, hardness/scratch resistance of polymer, stability of raw material, solvent compatibility, and cost.

1. Production of Nitrocellulose Membranes

Materials

Corning Gold Seal Slides (any plain glass slide may be used)

3M Optical Adhesives 8141, 8142, 8161 or 9483 (any of those listed may be used)

Pall Nitrocellulose Membrane (any manufacturers membrane may be substituted)

Rigid Tube Approximately ½ inch in Diameter (used to apply pressure to the adhesive)

Razor Blade

Tape

Process

A panel of slides was arranged in a rectangle composed of 7 columns and 2 rows. The slides were immobilized with standard lab tape to eliminate the possibility of movement during application of the adhesive and membrane. A segment of 2-sided optical adhesive was cut to match the rectangular panel and applied by first affixing one edge to the laboratory bench proximal and squared off with the rectangular grid such that release of tension would result in the adhesive flap dropping squarely on the slides. Tension is maintained with one hand as the other applies pressure to the contact edge of the adhesive with the rigid tube being applied by the opposite hand. The contact edge of the adhesive is moved forward by continued application of firm and even pressure across the tube's length as the opposite hand slowly releases pressure. The description given here is the manual manifestation of Nip Roll Lamination—a process fully characterized and familiar in industrial settings. The result is the bubble free application of an optically clear adhesive on which nitrocellulose membrane is applied.

The application of the nitrocellulose only requires a proper fit and gentle pressure smoothly applied across the surface with a hand or roller so to ensure proper adhesion. The panel of slides is then finished by segmentation with a razor blade to minimize any rough edges.

Other methods that are known in the art, such as pouring or casting membrane polymer matrices onto surfaces, can also be used with the present invention.

2. Microarray Layout, Membrane Transparification and Archival

| Materials: | |
|---|---|
| Deft Clear Lacquer | Cartesian Technologies Arrayer |
| Parks Clear Lacquer | RLS-view Instrument |
| 2-Butoxyethanol | |

Array Pattern

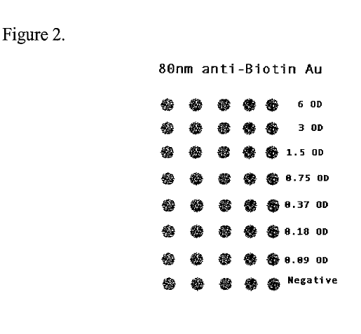

Nitrocellulose membranes as prepared above were spotted on a Cartesian Technologies arrayer in a rectangular array pattern as shown in FIG. 2 with 80 nm anti-biotin bound gold RLS particles.

Spotting was done in a formulation of 150 mM NaCl and 5% Bovine Serum Albumin. After arraying the slides were washed with distilled water. The highest and lowest concentrations of anti-biotin 80 nm Gold spotted were 6 OD and 0.09 OD respectively. These concentrations are quite low relative to what is achievable in a bioassay. The background levels observed on a 20 second scan on the RLS-view instrument (Genicon Sciences, San Diego, Calif.) were approximately 75 counts/sec for the best exemplary membrane archiving candidate. All membrane slides were dip coated and cured at Standard Temperature Pressure.

The following abbreviations were used as described in Table 1. They represent common usages and 3 transparifying/archival candidates chosen for this experiment.

TABLE 1

| Abbreviation | Definition | Refractive Index |
|---|---|---|
| Rkyv, Rkyvd, or, Rkyvng | Experimental usage of | Not Applicable |
| | Archive, Archived, and | |
| | Archiving. | |
| Au | Gold | Not Applicable |
| D100, or, d100 | Deft Clear Lacquer | 1.436 (liquid) |
| | 100% volume | |
| D50 EGME50, or, | Deft Lacquer 50% | 1.427 (liquid) |
| d50egme50 | 2-Butoxyethanol | |
| | 50% (v/v) | |
| P50 EGME50, or, | Parks Lacquer 50% | 1.422 (liquid) |
| p50egme50 | 2-Butoxyethanol | |
| | 50% (v/v) | |

TIF images of transparified membrane slides were captured on an RLS-view instrument. The spotting scheme is as indicated in FIG. 2. The arrays were applied in triplicate with duplicate slides prepared with each of the transparifying/archival candidates. Each of the images was held to the same screen stretch and instrument exposure time (20 seconds).

The Parks Clear Lacquer prepared with 50% v/v 2-Butoxyethanol shows indications of lower background and particulate inclusion; however, a second interesting point was observed. The spot intensities observed on the arrays coated with Deft Clear Lacquer 100% appear to be greater than for the other two coatings, perhaps attributable to the greater refractive index, or, thicker tegument produced by the undiluted Deft Lacquer.

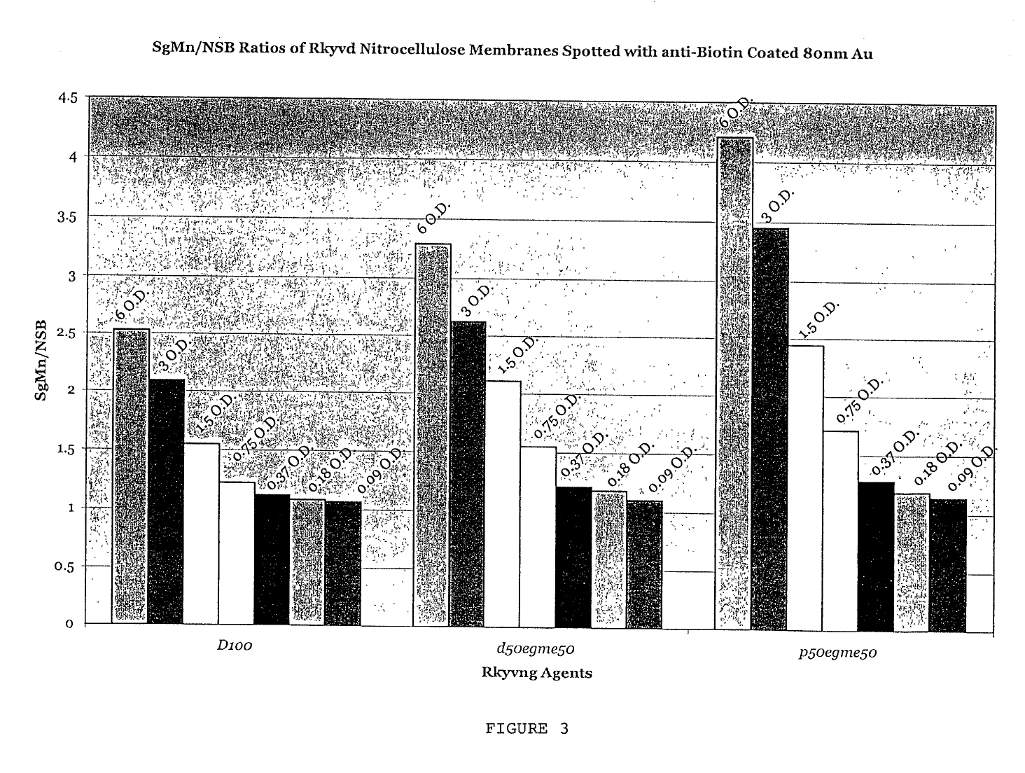

FIG. 3 is a graph showing signal to non-specific background ratios for 3 archiving materials on nitrocellulose membranes. The calculations were arrived at by dividing the signal mean of the spots observed by the average of negative spots in each array. This result is represented as the Average SgMn/NSB (Average Signal Mean Divided by Non-specific Background). The first and last bars in each set, corresponding to Rows 1 and 7 of FIG. 2, represent the highest and lowest anti-biotin 80 nm gold RLS particle densities, respectively. Row 8 is excluded as it is taken into account in the calculations (see Table 1 for the abbreviation definitions).

As indicated above, this example illustrates an effective one-step transparifying and archiving method. Additionally, a class of reagents is identified, for which candidate agents are readily available, inexpensive and easily modified in favor of more desirable properties (e.g., refractive index increase, viscosity reduction, volatility, etc.). These reagents and method are an extension of what is described in Example 1, with the exception that cellulose nitrate membranes are made transparent during application.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art will readily appreciate that the present invention is well adapted to carry out the objects and obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, using other sample devices and/or labeling techniques are all within the scope of the present invention. Thus, such additional embodiments are within the scope of the present invention and the following claims.

The invention illustratively described herein suitably may be practiced in the absence of any element or elements, limitation or limitations which is not specifically disclosed herein and/or may suitably be practiced in the presence of an additional element or elements, limitation or limitations. Thus, for example, in each instance herein any of the terms “comprising”, “consisting essentially of” and “consisting of” may be replaced with either of the other two terms for other embodiments. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Where a component or limitation is described with a variety of different possible numbers or dimensions associated with that component or limitation, in additional embodiments, the component or limitation is in a range specified by taking any two of the particular values provided as the endpoints of the range. The range includes the endpoints unless clearly indicated to the contrary.

Thus, additional embodiments are within the scope of the invention and within the following claims.

What we claim is:

1. A method for preserving a sample device having light scattering particle labels attached thereto, comprising
coating at least a portion of said sample device with an optically clear, solidifying solution, thereby providing a coated sample device.

2. The method of claim 1, wherein said sample device comprises a solid phase array.

3. The method of claim 1, wherein said sample device comprises a slide.

4. The method of claim 1, wherein said sample device comprises an array chip.

5. The method of claim 1, wherein said sample device comprises a microtiter plate.

6. The method of claim 1, wherein said sample device comprises a membrane.

7. The method of claim 1, further comprising storing said coated sample device under dark conditions.

8. The method of claim 1, wherein said sample device is a forensic sample device.

9. The method of claim 1, wherein said sample device is an identification sample device.

10. The method of claim 1, wherein said sample device is a clinical sample.

10. The method of claim 1, further comprising storing said coated sample device for a period of at least one month, wherein detectability of said light scattering particles remains substantially constant after said period.

11. A preserved sample device, comprising
a solid phase medium with light scattering particle labels attached thereto; and
an optically clear solid coating covering said light scattering particle labels.

12. The sample device of claim 11, wherein said sample device comprises a solid phase array.

13. The sample device of claim 11, wherein said sample device comprises a slide.

14. The sample device of claim 11, wherein said sample device comprises a microtiter plate.

15. The sample device of claim 11, wherein said sample device comprises an array chip.

16. The sample device of claim 11, wherein said sample device comprises a membrane.

17. A method for both transparifying and preserving a sample membrane with a single treatment, comprising
treating said membrane with a solidifying, non-membrane-dissolving, optically clear solution.

18. The method of claim 17, wherein said sample membrane has light scattering particle labels attached thereto.

19. The method of claim 18, wherein said sample membrane is associated with an optically clear solid phase support.

20. The method of claim 19, wherein said solid phase support is glass or plastic.

21. The method of claim 17, wherein said membrane is attached to or supported by a frame.

22. A method for reducing background light scattering in an analyte assay utilizing a sample device having light scattering particle labels attached thereto, comprising
coating at least a portion of said sample device with a solidifying, optically clear solution.

23. The method of claim 22, wherein said sample device comprises a solid phase array.

24. The method of claim 22, wherein said sample device comprises a slide.

25. The method of claim 22, wherein said sample device comprises an array chip.

26. The method of claim 22, wherein said sample device comprises a microtiter plate.

27. The method of claim 22, wherein said sample device comprises a membrane.

28. A method for enhancing specific detection of light scattering particle labels in an analyte assay utilizing a sample device having light scattering particle labels attached thereto, comprising
coating at least a portion of said sample device with an optically clear, solidifying solution, wherein said solution solidifies to provide a solid coating and said solid coating provides refractive index enhancement for detection of light scattered from said labels.

29. The method of claim 28, wherein said sample device comprises a solid phase array.

30. The method of claim 28, wherein said sample device comprises a slide.

31. The method of claim 28, wherein said sample device comprises an array chip.

32. The method of claim 28, wherein said sample device comprises a microtiter plate.

33. The method of claim 28, wherein said sample device comprises a membrane.

34. A method for delayed detection of analyte on a sample device having light scattering particle labels bound with analyte attached thereto and having an optically clear solid coating, comprising
detecting light scattered from said labels following storage for a period of at least one week, as an indication of the presence or amount or both of at least one analyte on said sample device,
wherein the detectability of said light is stable over said period.

35. The method of claim 34, wherein light scattered from said labels is also detected prior to said storage as an indication of the presence or amount or both of at least one analyte on said sample device.

36. The method of claim 34, wherein storage and detecting are performed a plurality of times.

37. The method of claim 34, wherein said sample device is stored for a period of at least one month prior to said detecting.

38. The method of claim 34, wherein the intensity of light scattered from said particles remains substantially constant following said storage.

39. The method of claim 34, further comprising washing said sample device before said detecting.

40. The method of claim 34, further comprising coating said sample device with an optically clear solidifying solution, thereby providing said optically clear solid coating.

41. The method of claim 42, further comprising storing said sample device for a period of at least one week prior to said detecting.

42. An assay method for detecting the presence or amount or both of an analyte on a sample device having light scattering particle labels bound with analyte attached thereto, comprising
illuminating said light scattering particle labels with light; and
detecting light scattered from said labels as an indication of the presence or amount or both of said analyte present on said sample device.

43. The method of claim 42, wherein said sample device comprises a solid phase array.

44. The method of claim 42, wherein said sample device comprises a slide.

45. The method of claim 42, wherein said sample device comprises an array chip.

46. The method of claim 42, wherein said sample device comprises a microtiter plate.

47. The method of claim 42, wherein said sample device comprises a membrane.

48. A kit comprising
a volume of an optically clear solidifying solution; and
a quantity of analyte-binding light scattering particle labels.

49. The kit of claim 48, wherein said light scattering particle labels comprise moieties that bind to analyte under binding conditions.

50. The kit of claim 48, wherein said light scattering particle labels comprise moieties that bind to analyte binding molecules under binding conditions.

51. The kit of claim 48, further comprising at least one sample device.

52. The kit of claim 51, wherein said sample device is a microarray.

53. The kit of claim 51, wherein said sample device is a slide.

54. The kit of claim 51, wherein said sample device is an array chip.

55. The kit of claim 51, wherein said sample device is a microtiter plate.

56. The kit of claim 51, wherein said sample device is a membrane.

57. A method for preparing a calibration slide, comprising
depositing predetermined amounts or dilutions of RLS particles at discrete locations on a sample device; and
coating said sample device with an optically clear solidifying solution following deposition of said particles.

58. The method of claim 57, wherein said sample device is selected from the group consisting of a chip, a slide, and a plate.

59. The method of claim 57, further comprising calibrating said calibration sample device to a master calibration standard.

60. A method of performing comparative analyte assays using light scattering particle labels bound with analyte, comprising
performing a calibrated assay of at least one sample device having analyte bound with said labels with an analyzer, thereby providing a first set of assay results;
performing a separate calibrated assay of at least one sample device having analyte bound with said labels with an analyzer, thereby providing at least a second set of assay results; and
comparing at least two of said set of assay results using scaled assay results.
